(19) United States
(12) Patent Application Publication
(10) Pub. No.: US 2006/0251748 A1
(43) Pub. Date: Nov. 9, 2006

(54) Novel strain of bacillus amyloliquefaciens and its use in obtaining fermented glycine max (L.) extract for inhibiting 15-lipoxygenase

(75) Inventor: Ming Shiang, Taipei City (TW)

Correspondence Address: HUSCH & EPPENBERGER, LLC, 190 CARONDELET PLAZA, SUITE 600, ST. LOUIS, MO, 63105-3441, US

(73) Assignee: MICROBIO CO., LTD., Taipei City (TW)

(21) Appl. No.: 11/131,686

(22) Filed: May 18, 2005

(30) Foreign Application Priority Data
May 5, 2005 (TW) 094114594

(51) Int. Cl. A61K 36/48; C12N 1/20

(52) U.S. Cl. 424/757; 435/252.31

(57) ABSTRACT

A novel microorganism designated as Bacillus amyloliquefaciens F5H-5 and a use of a fermented Glycine max (L.) extract prepared by fermenting an aqueous Glycine max (L.) extract with the microorganism in inhibiting 15-lipoxygenase are provided. In particular, the fermented Glycine max (L.) extract can be used in preventing and/or treating a disease in which 15-lipoxygenase inhibition is implicated in a subject, such as cardiovascular diseases.

### TECHNICAL FIELD

This invention relates to a novel microorganism designated as Bacillus amyloliquefaciens F5H-5 and a use of the microorganism in fermenting an aqueous Glycine max (L.) extract to obtain a fermented Glycine max (L.) extract, which is useful in inhibiting 15-lipoxygenase (LOX-15), preventing and/or treating a disease in which LOX-15 is implicated, such as cardiovascular diseases.

### BACKGROUND OF THE INVENTION

Lipoxygenases (LOX) are nonheme iron-containing enzymes that catalyze the oxygenation of certain polyunsaturated fatty acids such as lipoproteins. Several different types of lipoxygenase enzymes, e.g. LOX-5, LOX-12 and LOX-15, are known, each having a characteristic oxidation action. LOX-15 catalyzes the oxygenation of arachidonic and linoleic acids and has been implicated in the oxidative modification of low-density lipoproteins (LDL). Many researches reported that the LOX-15 is associated with coronary artery disease and atherosclerosis (Shen et al., J. Clin. Invest. 1996, Vol. 98, No. 10, pp. 2201-2208; Hiltunen et al., Circulation 1995, Vol. 92 (11), pp. 3297-3303; Ravalli et al., 1995, Arteriosclerosis, Thrombosis and Vascular Biology, Vol. 15, No. 3, pp. 340-348; and Kuhn et al., 1997, J. Clin. Invest., Vol. 99, No. 5, pp. 888-893), cancer and inflammatory diseases (U.S. Pat. No. 6,001,866; Mogul et al., 2001, Biochemistry, 40, 4391-4397; Walther et al., 1999, Molecular Pharmacology, 56: 196-203; and Kamitani et al., 1998, the Journal of Biological Chemistry, Vol. 273, No. 34, pp. 21569-21577), and the immune response (Kruisselbrink et al., 2001, Clin Exp Immunol, 126:2-8). Therefore, a substance having an efficacy in inhibiting LOX is useful as an agent for preventing or treating diseases associated with LOX.

Soybeans are one concentrated source of isoflavones in human diet. They also contain many other compounds including saponins, phytosterols, soybean phytates, protease inhibitors, phenolic acids, complex sugars, boron, lecithin, omega-3 fatty acids and folic acid. They can impart health benefits. Many traditional eastern foods, such as tempeh and natto, are produced from the fermentation of soybeans. For example, tempeh is produced by fermenting soybean with Rhizopus oligosporus, R. oryzae, R. arrihizus and R. stolonifer Natto is produced by fermenting soybean with Bacillus natto. The traditional fermented foods can be used as a superior protein origin.

U.S. Pat. No. 6,685,973 B1 discloses a fermented Glycine max (L.) extract, which is prepared by fermenting an aqueous Glycine max (L.) extract with at least one lactic acid bacteria together with at least one yeast, and said to be effective in inhibiting LOX-15. It does not disclose or suggest that a fermented Glycine max (L.) extract having LOX-15 inhibiting activity can be prepared with any other microorganisms.

### REFERENCES

Adlercreuz, H. et al., Evaluation nutrition, intestinal microflora and prevention of cancer: a hypothesis, Proc. Soc. Exp. Biol. Med., 217:241-246 (1998).
Breimer L H. Ionizing radiation-induced mutagenesis, Br J Cancer, 57:6-18 (1998).
Briehl, M. M. et al., Modulation of the antioxidant defense as a factor in apoptosis, Cell Death Differ., 3:63-70 (1996).
Bruce et al., A Spectrophotometric Microtiter-Based Assay for the Detection of Hydroperoxy Derivatives of Linoleic Acid, Analytical Biochemistry, Vol. 201, pp. 375-380 (1992).
Chemoprevention Working Group to the American Association for Cancer Research, Cancer Res. 59:4743-4758 (1999).
Cohen, L. A. et al., Effect of intact and isoflavone-depleted soybean protein on NMU-induced rat mammary tumorigenesis, Carcinogenesis, 2: 929-935 (2000).
Dwyer, J. T. et al., Tofu and soybean drinks contains phytoestrogenes, J. Am. Diet Assoc., 94: 739-743 (1994).
Ghibelli, L. et al., Rescue of cells from apoptosis by inhibition of active GSH extrusion, FASEB J., 12: 479-486 (1998).
Greenwald, P. et al., Chemoprevention, CA-Cancer J. Clin., 45:31-49 (1995).
Hiltunen et al., Circulation 1995, Vol. 92 (11), pp. 3297-3303.
Hong, W. K. et al., Recent advances in chemoprevention of cancers, Science, 278:1073-1077 (1993).
Hutchins, A. M. et al., Urinary isoflavoneoid phytoestrogen and lignan excretion after consumption of fermented and unfermented soybean products, J. Am. Diet Assoc., 95:545-551 (1995).
Ikeda, Y. et al., The molecular basis of brain injury and brain edema: the role of oxygen free radicals, Neurosurgery, 27:1-11 (1990).
Keisari, Y. et al., A simple colorimetric method for the measurement of hydrogen peroxide produced by cells in culture, J. Immunol Methods., 38:161-170 (1980).
Kelloff, G. J., Approaches to the development and marketing approval of drugs that prevent cancer, Cancer Epidermiol. Biomarkers Pre., 4:1-10 (1995).
Kontos H A et al., Oxygen radicals in brain injury, CNS Trauma, 3:257-63 (1986).
Messina, M. et al., Soybean intake and cancer risk: a review of the in vitro and in vivo data, Nutr. Cancer, 21:113-131 (1994).
Nout, M. J. R. et al., Recent development in temphe research, J. Appl. Bacteriol., 69:609-633 (1990).
Plamer, H. J. et al., Reactive oxygen species and antioxidants in signal transduction and gene expression, Nutr. Rev, 55: 353-361 (1997).
Robak J. et al., Flavonoids are scavengers of superoxide anions, Biochemical Pharmacology, 37(5):837-41 (1988).
Shao, Z. M. et al., Genistein exerts mutiple suppressive effects on human breast carcinoma cells, Cancer Res., 58:4851-4857 (1998).
Steinberg D. et al, Beyond cholesterol: modifications of low-density lipoprotein that increase its atherogenicity, N Engl J Med, 320:915-24 (1989).
Toshiki, Y. et al., Mechanism of catechin chemiluminescence in the presence of active oxygen, J. Biolumin. Chemilumin., 11:131-136(1996).
Wang, H. et al., Isoflavone content of commercial soys foods, J. Agric. Food Chem., 42:1666-1673 (1994).

### SUMMARY OF THE INVENTION

One object of the invention is to provide a new microorganism, Bacillus amyloliquefaciens F5H-5, which is capable of fermenting an aqueous Glycine max (L.) extract to obtain a fermented Glycine max (L.) extract for inhibiting LOX-15.

Another object of the invention is to provide a use the microorganism of the present invention for the preparation of a fermented Glycine max (L.) extract for inhibiting LOX-15 and preventing and/or treating diseases wherein LOX-15 is implicated.

A further object of the invention is to provide a method for inhibiting LOX-15 in a subject, which comprises administering an effective amount of a fermented Glycine max (L.) extract produced by the microorganism of the present invention.

Still another object of the invention is to provide a composition for inhibiting LOX-15, comprising an effective amount of a fermented Glycine max (L.) extract, which is prepared by fermenting an aqueous Glycine max (L.) extract with the microorganism of the present invention.

The fermented Glycine max (L.) extract of the invention can be used in preventing and/or treating a disease in which LOX-15 inhibition is implicated, such as cardiovascular diseases, in a subject.

### BRIEF DESCRIPTION OF THE DRAWINGS

FIG. 1 shows the effect of a fermented black soybean extract on the lesion formation in apoE-deficient mice.

FIG. 2 shows the staining of aortic lesions from apoE-deficient mice.

### DETAILED DESCRIPTION OF THE INVENTION

The invention provides a new microorganism strain for fermenting a Glycine max (L.) extract to produce a fermented Glycine max (L.) extract useful in inhibiting LOX-15. The applicant surprisingly obtained a novel microorganism, Bacillus amyloliquefaciens strain F5H-5, through the isolation from bean paste samples. This strain has been deposited with the China Center for Type Culture Collection (CCTCC) under the provisions of the Budapest Treaty on ______, under Accession No: ______.

The invention also provides a method for the production of a fermented Glycine max (L.) extract useful in inhibiting LOX-15 comprising contacting an aqueous Glycine max (L.) extract with Bacillus amyloliquefaciens F5H-5.

The invention further provides a method for treating or reducing the risk of cardiovascular disease in a subject through the inhibition of LOX-15, which comprises administering an effective amount of a fermented Glycine max (L.) extract to the subject, wherein the fermented Glycine max (L.) extract is prepared by fermenting an aqueous Glycine max (L.) extract with Bacillus amyloliquefaciens F5H-5.

The present invention also provides a composition comprising the fermented Glycine max (L.) extract of the invention. The composition of the present invention can be used as a pharmaceutical composition or as a food composition.

The fermented Glycine max (L.) extract and composition of the present invention can be used in preventing and/or treating a disease in which LOX-15 is implicated, such as cardiovascular diseases (e.g. atherosclerosis).

### Process for Producing the Fermented Soybean Extract

The fermented Glycine max (L.) extract is made by fermentation of an aqueous Glycine max (L.) extract with Bacillus amyloliquefaciens F5H-5, followed by sterilization, e.g. by heat, of the fermented liquid with optional filtration and concentration.

According to the invention, the preferred Glycine max (L.) used in the preparation of the fermented Glycine max (L.) extract is selected from the group consisting of soybean and black soybean. More particularly, the fermented Glycine max (L.) extract of the invention is the fermented soybean extract or fermented black soybean extract.

Preferably, an aqueous extract of non-genetically modified organic Glycine max (L.) of selected grade is used as a starting material. After fermentation, the fermented liquid is sterilized, e.g. by heat or irradiation, preferably by heat, to obtain a sterilized liquid. Preferably, the sterilized liquid is filtered or centrifuged, preferably centrifuged, to remove most or all of the dead microbes to obtain the fermented Glycine max (L.) extract. More preferably, the centrifugation step is followed by removal of some of the water from the supernatant to concentrate the fermented liquid to obtain a concentrated fermented Glycine max (L.) extract. Optionally, the fermented Glycine max (L.) extract can be dried, e.g. via lyophilization, to obtain the fermented Glycine max (L.) extract in a powdered form.

The process can be carried out by mixing organic Glycine max (L.) powder (1-10% w/w) with distilled water to obtain a Glycine max (L.) extract. According to the present invention, all known carbon and nitrogen sources can be added to the Glycine max (L.) extract. The preferred carbon source includes, but not limited to, starch, sugar, glucose, maltose, sucrose, glycerol, and combination thereof; the preferred nitrogen source includes, but not limited to, yeast extract, glutamic acid, ammonium chloride, potassium nitrate, and combination thereof. The added amount of carbon source is from 0.5-15% (w/w), preferably from 1-10% (w/w), and more preferably from 1-4% (w/w). The added amount of nitrogen source is from 0.25-5% (w/w), preferably from 0.5-3% (w/w), and more preferably from 0.5-2% (w/w).

Bacillus amyloliquefaciens F5H-5 is incubated in Nutrient Broth as a seed culture. The seed culture is transferred to the Glycine max (L.) extract and incubated at 30° C. for 50-80 hours. The fermented extract is heat-sterilized and centrifuged, and the supernatant is collected. The water content of the supernatant may be removed by any conventional methods to obtain a fermented Glycine max (L.) extract in a concentrated or powdered form.

Use in the Inhibition of LOX-15

LOX-15 is nonheme iron-containing enzyme that catalyzes the oxygenation of certain polyunsaturated fatty acids such as lipoproteins. It is known that compounds inhibit the action of LOX-15 enzyme are useful in the treatment or alleviation of inflammatory diseases, allergy, cardiovascular diseases and immune disorders in mammals including humans.

Studies in the invention have demonstrated that the fermented Glycine max (L.) extract of the present invention can inhibit LOX-15 superior to the known fermented soybean foods and the unfermented soybean.

Use as an Antioxidant

The fermented Glycine max (L.) extract has prominent antioxidant and free radical scavenger activities. The fermented Glycine max (L.) extract can remove superoxide-free radicals, e.g., O₂⁻, H₂O₂ and ROO, and can act as an antioxidant for unsaturated fatty acid and fat. The fermented Glycine max (L.) extract has a prominent ability to eliminate hyper oxygen anions to protect the cell from oxidative injury and change free radicals to harmless substances with an energy decreasing procedure.

Use as Agent Against Cardiovascular Diseases

Many studies shows that the inhibitors of LOX-15 in the treatment and prevention of inflammation and atherosclerosis (Cornicelli et al., U.S. Pat. No. 6,001,866; Bocan et al., Atherosclerosis, 136, 203-216, 1998 and Hiltunen et al., Circulation, 92 (1), 1 Dec. 1995, pp. 3297-3303). It is expected that the fermented Glycine max (L.) extract is useful for preventing and/or treating cardiovascular diseases, such as atherosclerosis.

Administration of the Fermented Glycine max (L.) Extract

In accordance with this invention, the fermented Glycine max (L.) extract may be administered alone or in combination with a pharmaceutically acceptable carrier, diluent and/or excipient, as a composition. Preferably, the fermented Glycine max (L.) extract is fermented soybean extract or fermented black soybean extract. The fermented Glycine max (L.) extract may be administered at a dose of about 0.1 to 20 mg/kg body weight, with a maximum dose of 50 mg per person per administration. Preferably, the dose of the fermented Glycine max (L.) extract is 1 to 10 mg/kg, more preferably 2 to 5 mg/kg, body weight of the subject. These doses are based on the fermented Glycine max (L.) extract in the powdered form, but appropriate doses of the fermented Glycine max (L.) extract in the unconcentrated form or concentrated form can be calculated accordingly. The dose can be adjusted based on the health condition of the subject or the disease to be prevented or treated.

This invention will now be described with reference to the following non-limiting examples.

### EXAMPLE 1

Isolation and Characterizations of Bacillus amyloliquefaciens F5H-5

Bean paste sample (1 g) was stirred and diluted in 100 ml sterilized water. The obtained suspension was heated at 80° C. for 5 minutes, and then streaked on Nutrient Agar plates. The plates were incubated at 30° C. for 24 hours. Each Colony occurring on the plates was further streaked on a Nutrient Agar plate, and this step was repeated twice to isolate a pure strain of microorganism. Stain F5H-5 examined by the LOX-15 activity test shown below was selected from 62 isolated strains.

Stain F5H-F was identified to be a Gram-positive, endospore-producing, and aerobic bacterium, which has catalyst but without oxygenase. Based on 16S rDNA partial sequencing analysis, F5H-F has an identity of up to 97% with Bacillus amyloliquefaciens, B. atrophaeus, B. licheniformis, B. mojavensis, B. subtilis, and B. vallismortis. The fatty acid contents of F5H-F are shown in Table 1.

TABLE 1
Analysis of cellular fatty acids within F5H-F

| Name | % |
|---|---|
| 14:0 ISO | 1.48 |
| 15:0 ISO | 14.2 |
| 15:0 ANTEISO | 37.66 |
| 16:0 ISO | 4.39 |
| 16:1 w10c | 1.85 |
| 16:0 | 6.53 |
| ISO 17:1 w10c | 2.08 |
| 17:0 ISO | 15.54 |
| 17:0 ANTEISO | 15.13 |
| 18:0 | 1.14 |

Data base similarity index: Bacillus subtilis 0.687

The DNA of F5H-F was used as a probe to hybridize with the DNA of 6 standard Bacillus strains. The results are shown in Table 2.

TABLE 2
The results of DNA hybridization

| Standard strain | Similarity |
|---|---|
| Bacillus subtilis BCRC10255 | 26.6% |
| Bacillus amyloliquefaciens BCRC11601 | 92.4% |
| Bacillus licheniformis BCRC11702 | 17.7% |
| Bacillus atrophaeus BCRC17123 | 32.2% |
| Bacillus mojavensis BCRC17124 | 26.0% |
| Bacillus vallismortis BCRC17138 | 25.2% |

Based on the DNA hybridization results, we believe that strain F5H-F should be classified as Bacillus amyloliquefaciens.

### EXAMPLE 2

LOX-15 Inhibition Tests

The assay was performed in a 96-well microtiter plate based on the procedures disclosed by Bruce et al. (Analytical Biochemistry, 1992, Vol. 201, pp. 375-380). Each well contains 40 μl of substrate solution (containing linoleic acid as the substrate of LOX-15) and 5 μl of diluted unfermented or fermented black soybean extract. After 5 minutes at 4° C., 5 μl of LOX-15 (100 U) isolated from rabbit reticulocytes was added to each well of the plate. The plate was then kept at 4° C. for 10 minutes followed by the addition of 100 μl of LMB color reagent. After 10 minutes at room temperature, the OD₆₆₀ values of the samples were determined by a microtiter plate reader.

TABLE 3
The results of LOX-15 inhibition tests

| | Black Soybean | |
|---|---|---|
| Samples | concentration (%) | LOX-15 Inhibition Rate (%) |
| F5H-F | 2 | 24.3 ± 3.8 |
| | 4 | 46.6 ± 4.7 |
| | 6 | 61.2 ± 8.9 |
| Unfermented Black | 2 | 15.8 |
| Soybean | 4 | 32.1 |
| | 6 | 46.4 |

### EXAMPLE 3

In Vivo Animal Studies

Atherosclerosis is a disease with complex etiology. Several risk factors, including hyperlipidemia, oxidative stress, and inflammation, are implicated in the development of atherosclerotic lesions. Agents that can reduce blood cholesterol, lipid peroxidation or vascular inflammation reaction are considered as a potential regimen for the treatment of this disease. To evaluate the potential of a therapeutic agent in vivo, apoe-deficient mice, which have been shown to develop spontaneously hypercholesteroemia and atherosclerosis lesions with the features similar to human patients, were used to assay the efficacy of the fermented product in the treatment of cardiovascular diseases.

At the age of 3 months, 20 apoE-deficient mice were divided into 2 groups: the control group, wherein the mice were fed with chow diet, and the test group, wherein the mice were fed with diet containing the dried fermented black soybean extract product at a dosage of 0.8 mg/animal/day of for 3 months.

At indicated time intervals, blood samples were collected from all mice through tail veins. Plasma concentration of cholesterol and triglyceride were determined using Sigma INFINITY™ cholesterol reagent (No. 401-100P) and INFINITY™ triglyceride reagent (No. 343-25P). As shown in Tables 4 and 5, the treatment with the fermented black soybean extract product of the present invention significantly decreased the increasing rate of the plasma cholesterol level (p<0.05), and the increasing rate of the total plasma triglyceride level in mice receiving the fermented black soybean extract product of the present invention was also significantly reduced (p<0.05).

TABLE 4
The plasma levels of cholesterol of apoE-deficient mice

Treatment
Cholesterol (mg/dL)

| Time (M) | Control | Fermented Product |
|---|---|---|
| 0 | 393.8 ± 48.6 | 376.3 ± 58.7 |
| 1 | 457.3 ± 63.1 | 404.1 ± 60.2 |
| 2 | 479.4 ± 47.5 | 417.2 ± 34.4 |
| 3 | 577.3 ± 81.4 | 465.4 ± 66.0 |

TABLE 5
The plasma levels of triglyceride of apoE-deficient mice

Treatment
Triglyceride (mg/dL)

| Time (M) | Control | Fermented Product |
|---|---|---|
| 0 | 92.04 ± 20.95 | 76.69 ± 12.39 |
| 1 | 42.80 ± 11.95 | 35.34 ± 5.23 |
| 2 | 50.34 ± 10.10 | 43.74 ± 11.9 |
| 3 | 86.67 ± 23.51 | 49.26 ± 9.31 |

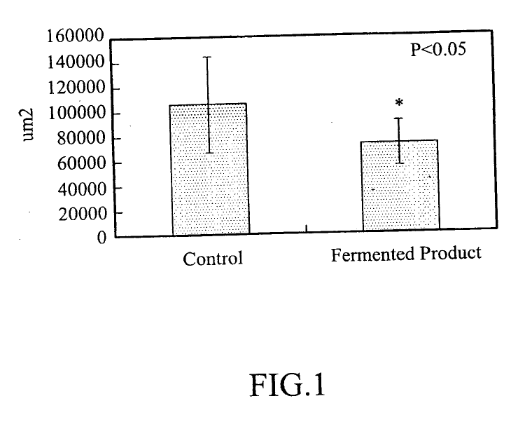

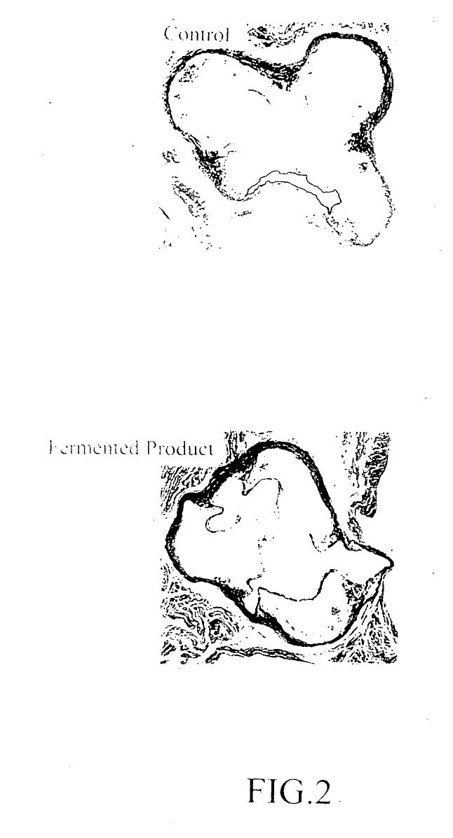

Animals were then sacrificed and the aortic sinus of each mouse was collected. For the quantification of the atherosclerotic lesions, 50 serial sections from the aortic sinus of each mouse were collected. A total of 10 sections samples from every 5 consecutive sections were subjected to trichrome staining, and photomicrographs were taken. The cross-sectional area of each photomicrograph was analyzed by a computer imaging graphic software. The lesion size was then calculated from the average of the area quantified from the 10 sections. It is found that the lesion of atherosclerosis in mice receiving the fermented black soybean extract product of the present invention was significantly reduced in comparison with the control mice (p<0.02) (see FIGS. 1 and 2).

According to the above results, the fermented black soybean extract product of the present invention indeed has an efficacy in decreasing the level of plasma cholesterol and plasma triglyceride, and treating cardiovascular disease, such as arteriosclerosis.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An isolated Bacillus amyloliquefaciens F5H-5 having an accession number CCTCC ______.

2. A method for the production of a fermented Glycine max (L.) extract useful in inhibiting 15-lipoxygenase, which comprises fermenting an aqueous Glycine max (L.) extract with the isolated Bacillus amyloliquefaciens F5H-5 according to claim 1.

3. The method of claim 2, wherein the Glycine max (L.) extract is a black soybean extract.

4. The method of claim 2, which further comprises removing the water content to obtain a concentrated or powdered form of the fermented Glycine max (L.) extract.

5. A method for treating or reducing the risk of cardiovascular disease in a subject through the inhibition of 15-lipoxygenase, comprising administering an effective amount of a fermented Glycine max (L.) extract obtained according to the method of claim 2 to the subject.

6. The method of claim 5, wherein the fermented Glycine max (L.) extract is a fermented black soybean extract.

7. The method of claim 5, wherein the fermented Glycine max (L.) extract is in a concentrated or powdered form.

8. The method of claim 5, wherein said cardiovascular disease is atherosclerosis.

9. A composition for treating or reducing the risk of cardiovascular disease in a subject through the inhibition of 15-lipoxygenase, comprising an effective amount of a fermented Glycine max (L.) extract obtained according to the method of claim 2.

10. The composition of claim 9, wherein the fermented Glycine max (L.) extract is a fermented black soybean extract.

11. The composition of claim 9, wherein the fermented Glycine max (L.) extract is in a concentrated or powdered form.

12. The composition of claim 9, wherein said cardiovascular disease is atherosclerosis.

13. The composition of claim 9, which is a pharmaceutical composition or a food composition.
